(19) United States
(12) Patent Application Publication
(10) Pub. No.: US 2012/0027169 A1
(43) Pub. Date: Feb. 2, 2012

(54) RADIOLOGICAL IMAGE RADIOGRAPHING AND DISPLAYING METHOD AND APPARATUS

(75) Inventor: Hiroki NAKAYAMA, Ashigarakami-gun (JP)

(73) Assignee: FUJIFILM CORPORATION, Tokyo (JP)

(21) Appl. No.: 13/191,149

(22) Filed: Jul. 26, 2011

(30) Foreign Application Priority Data
Jul. 28, 2010 (JP) 2010-168938

(51) Int. Cl. A61B 6/02

(52) U.S. Cl. 378/41

(57) ABSTRACT

A stereoscopic image radiographing apparatus performing both an equal-magnification radiographing operation and an enlarged radiographing operation displays a stereoscopic image with a magnitude of parallax suitable for both radiographing operations without changing two focal positions of radiation. Two radiological images are relatively moved and displayed in a parallax direction which is obtained by projecting an extending direction of a straight line connecting two focal positions onto the corresponding radiological image based on the angle formed by two radiographing directions of two radiological images constituting the stereoscopic image and the enlargement ratio thereof.

### BACKGROUND OF THE INVENTION

1. Field of the Invention

The present invention relates to radiological image radiographing and displaying method and apparatus in which a subject is irradiated with radiation from two different radiographing directions, radiological images are detected in the radiographing directions, and a stereoscopic image is displayed using the two detected radiological images, and more particularly, to radiological image radiographing and displaying method and apparatus in which a radiological image is detected by changing a distance between a subject and a radiological image detector depending on an enlargement ratio of the radiological image.

2. Description of the Related Art

It has been known that an image can be viewed stereoscopically using parallax by combining and displaying plural images. Such an image (hereinafter, referred to as a stereoscopic image or a stereo image) that can be viewed stereoscopically is generated based on plural images with parallax acquired by radiating the same subject from different directions.

Moreover, such way of generating stereoscopic image is utilized not only in the field of digital cameras and televisions but also in the field of capturing a stereoscopic radiological image. That is, a test subject is irradiated with radiation from different directions, the radiation passing through the test subject is detected by a radiological image detector to acquire plural radiological images having parallax, and a stereoscopic image is generated based on the radiological images. By generating a stereoscopic image in this way, it is possible to observe a radiological image with a sense of depth and thus to observe a radiological image more suitable for diagnosis.

On the other hand, rather than equal-magnification radiographing in which a radiographing operation is performed in a state where a radiological image detector and a subject comes in close contact with each other, enlargement radiographing in which an enlarged image is captured by performing a radiographing operation in a state where the radiological image detector and the subject are separated from each other has been used.

### SUMMARY OF THE INVENTION

Here, in a radiological image radiographing apparatus radiographing the stereoscopic image, it is considered that both the equal-magnification radiographing and the enlargement radiographing are carried out. For example, when the focal positions of a radiation source at the time of radiographing two radiological images are fixed to the direction of 0° and the direction of +θ°, two radiological images projected onto a detection plane (projection plane) of the radiological image detector in the normal radiographing (equal-magnification radiographing) are P01 and P02 shown in FIG. 7A and two radiological images projected on the detection plane (projection plane) of the radiological image detector in the enlargement radiographing are P01′ and P02′ shown in FIG. 7A. That is, when two focal positions are fixed, the magnitude of parallax between two radiological images in the enlarged radiographing becomes larger than the magnitude of parallax between two radiological images in the normal radiographing, thereby causing a problem in that the stereoscopic image cannot be appropriately viewed stereoscopically. Particularly, since the peripheral region has the large magnitude of parallax, it is very difficult to view the image stereoscopically. Here, the magnitude of parallax means the magnitude of shift between two images.

Therefore, in JP-H09-187447A (JP1997-187447A), an apparatus performing both the equal-magnification radiographing and the enlarged radiographing is proposed as the radiological image radiographing apparatus radiographing a stereoscopic image. A method of changing the distance between two focal positions in the equal-magnification radiographing and the enlarged radiographing is proposed to set the magnitudes of parallax suitable for both radiographing operations.

However, as described in JP-H09-187447A (JP1997-187447A), when the distance between two focal positions is changed, it is necessary to set the distance between two focal positions to be smaller than that in the normal radiographing at the time of performing the enlarged radiographing as shown in FIG. 7B. However, in order to set the distance between two focal positions to a distance suitable for the enlarged radiographing in this way, it is necessary to control the movement of the radiation source with the accuracy of 1°. In this case, the structure of the image radiographing apparatus is complicated, thereby raising the cost. Particularly, in an image radiographing apparatus radiographing a phase-contrast image, since it is necessary to set the distance between the radiological image detector and the radiation source to be relatively large, it is necessary to control the movement of the radiation source with higher accuracy as much.

The present invention has been made in view of the above-mentioned problems and an object of the invention is to provide a radiological image radiographing and displaying method and apparatus in which both an equal-magnification radiographing operation and an enlarged radiographing operation are performed, which can display a stereoscopic image with a magnitude of parallax suitable for both radiographing operations without changing two focal positions of radiation.

According to an aspect of the present invention, there is provided a radiological image radiographing and displaying method of irradiating a subject with radiation radiated from two focal positions from two different radiographing directions, detecting a radiological image in each radiographing direction resulting from the irradiation with the radiation by the use of a radiological image detector, and displaying a stereoscopic image using the two detected radiological images, wherein the distance between the subject and the radiological image detector is changed depending on an enlargement ratio of the radiological images to detect the radiological images, and the two radiological images are relatively moved and displayed in a parallax direction which is obtained by projecting an extending direction of a straight line connecting the two focal positions onto the corresponding radiological image based on the angle formed by the two radiographing directions and the enlargement ratio.

According to another aspect of the present invention, there is provided a radiological image radiographing and displaying method of irradiating a subject with radiation radiated from two focal positions in two different radiographing directions, detecting a radiological image in each radiographing direction resulting from the irradiation with the radiation by the use of a radiological image detector, and displaying a stereoscopic image using the two detected radiological images, wherein the distance between the subject and the radiological image detector is changed depending on an enlargement ratio of the radiological images to detect the radiological images, and the radiological image detector is relatively moved in a parallax direction, which is obtained by projecting an extending direction of a straight line connecting the two focal positions onto the corresponding radiological image based on the angle formed by the two radiographing directions and the enlargement ratio, to capture the radiological images.

According to yet another aspect of the present invention, there is provided a radiological image radiographing and displaying apparatus including: a radiation irradiating unit that irradiates a subject with radiation radiated from two focal positions from two different radiographing directions; a radiological image detector that detects a radiological image in each radiographing direction resulting from the irradiation with the radiation from the radiation irradiating unit; a distance changing mechanism that changes the distance between the subject and the radiological image detector depending on an enlargement ratio of the radiological images; and a display unit that displays a stereoscopic image using two radiological images detected by the radiological image detector, wherein the display unit relatively moves the two radiological images in a parallax direction which is obtained by projecting an extending direction of a straight line connecting the two focal positions onto the corresponding radiological image based on the angle formed by the two radiographing directions and the enlargement ratio, and the display unit displays the moved two radiological images.

According to yet another aspect of the present invention, there is provided a radiological image radiographing and displaying apparatus including: a radiation irradiating unit that irradiates a subject with radiation radiated from two focal positions from two different radiographing directions; a radiological image detector that detects a radiological image in each radiographing direction resulting from the irradiation with the radiation from the radiation irradiating unit; a distance changing mechanism that changes the distance between the subject and the radiological image detector depending on an enlargement ratio of the radiological images; a display unit that displays a stereoscopic image using two radiological images detected by the radiological image detector; and a moving mechanism that relatively moves the radiological image detector in a parallax direction which is obtained by projecting an extending direction of a straight line connecting the two focal positions onto the corresponding radiological image based on the angle formed by the two radiographing directions and the enlargement ratio.

The radiological image radiographing and displaying apparatus may further include an image shifting unit that performs a shifting process of relatively shifting the two radiological images in the parallax direction based on the angle formed by the two radiographing directions and the enlargement ratio.

The radiological image radiographing and displaying apparatus may further include a display controlling unit that controls the relative shifting and displaying of the two radiological images in the parallax direction based on the angle formed by the two radiographing directions and the enlargement ratio when displaying the two radiological images on the display unit.

The radiological image radiographing and displaying apparatus may further include an image reducing unit that raises a reduction ratio of the two radiological images with the raising of the enlargement ratio.

The image shifting unit may perform the shifting process so that the magnitude of parallax between the two radiological images when the enlargement ratio is larger than 1 approaches the magnitude of parallax between the two radiological images when the enlargement ratio is approximately an equal magnification.

The display control unit may control the relative shifting and displaying of the two radiological images so that the magnitude of parallax between the two radiological images when the enlargement ratio is larger than 1 approaches the magnitude of parallax between the two radiological images when the enlargement ratio is approximately an equal magnification.

The moving mechanism may move the radiological image detector so that the magnitude of parallax between the two radiological images when the enlargement ratio is larger than 1 approaches the magnitude of parallax between the two radiological images when the enlargement ratio is approximately an equal magnification.

The radiological image radiographing and displaying apparatus may further include a radiographing mode instruction receiving unit that receives an instruction of a normal radiographing mode in which the enlargement ratio is approximately an equal magnification and an enlarged radiographing mode in which the enlargement ratio is larger than 1.

The distance changing mechanism may move the radiological image detector relative to the subject.

In the radiological image radiographing and displaying method and apparatus according to the present invention, since two radiological images are relatively moved and displayed in the parallax direction based on the angle formed by two radiographing directions and the enlargement ratio, it is possible to display a stereoscopic image with a magnitude of parallax suitable for both the equal-magnification radiographing and the enlarged radiographing without changing the distance between two focal positions of radiation.

Even by relatively moving the radiological image detector in the parallax direction based on the angle formed by two radiographing directions and the enlargement ratio and radiographing the radiological images, it is possible to achieve the same advantages as described above.

### BRIEF DESCRIPTION OF THE DRAWINGS

FIG. 1 is a diagram schematically illustrating the configuration of a breast image radiographing and displaying system employing a radiological image radiographing and displaying apparatus according to an embodiment of the present invention.

FIG. 2 is a diagram illustrating an arm section of the breast image radiographing and displaying system shown in FIG. 1 as viewed from the right side of FIG. 1.

FIG. 3 is a block diagram schematically illustrating the internal configuration of a computer of the breast image radiographing and displaying system shown in FIG. 1.

FIG. 4 is a flowchart illustrating the operation of the breast image radiographing and displaying system employing the radiological image radiographing and displaying apparatus according to the embodiment of the present invention.

FIG. 5 is a diagram schematically illustrating radiological images P1 and P2 in a normal radiographing mode, radiological images P3 and P4 in an enlarged radiographing mode, and radiological images P3′ and P4′ having been subjected to a reduction process.

FIG. 6 is a diagram illustrating a radiological image radiographing and displaying apparatus according to another embodiment of the present invention.

FIG. 7 is a diagram illustrating normal radiographing and enlarged radiographing for a stereo image according to the background art.

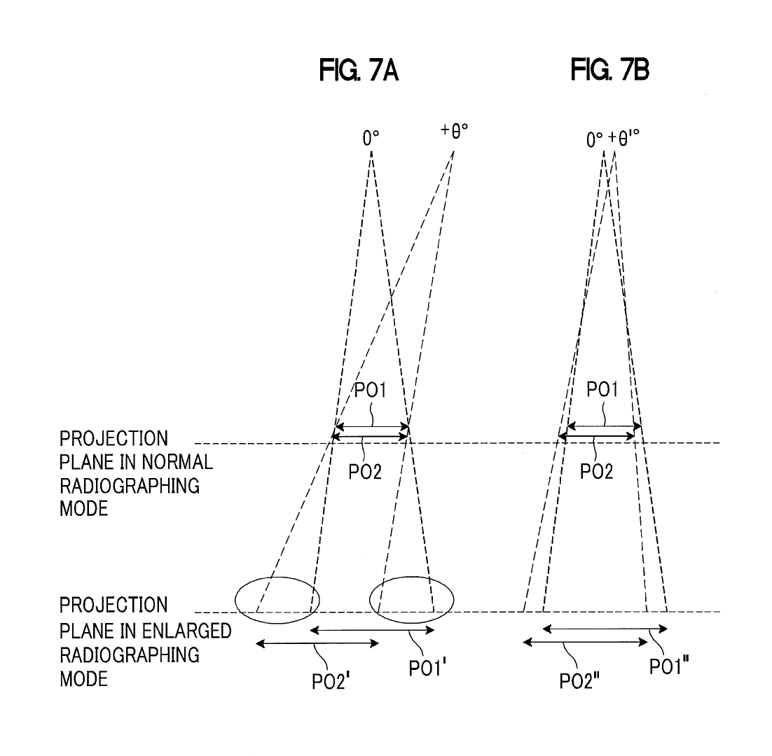

### DESCRIPTION OF THE PREFERRED EMBODIMENTS

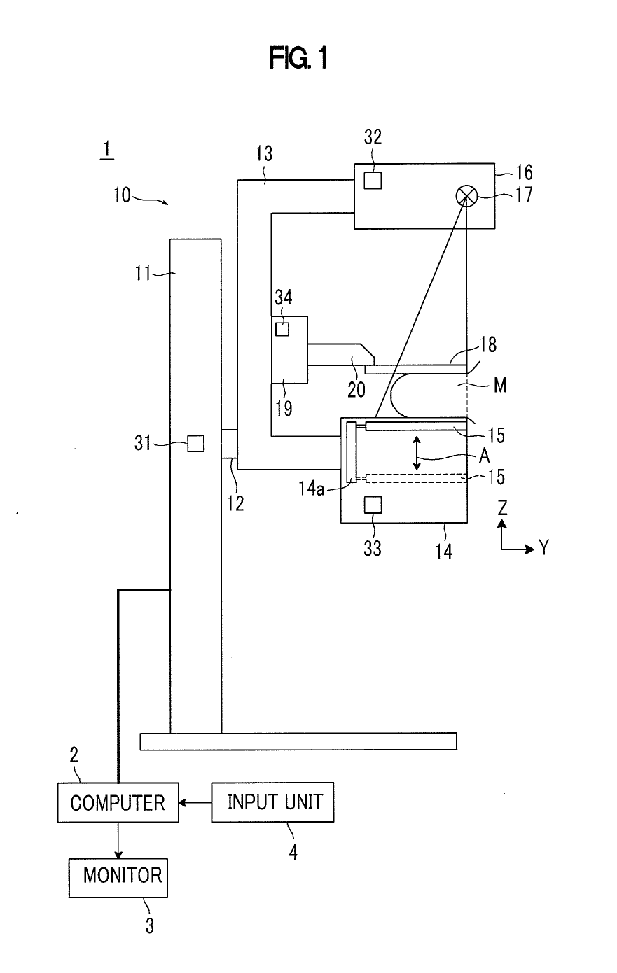

Hereinafter, a breast image radiographing and displaying system employing a radiological image radiographing and displaying apparatus according to an embodiment of the present invention will be described with reference to the accompanying drawings. FIG. 1 is a diagram schematically illustrating the entire configuration of the breast image radiographing and displaying system according to this embodiment.

As shown in FIG. 1, the breast image radiographing and displaying system 1 includes a breast image radiographing apparatus 10, a computer 2 connected to the breast image radiographing apparatus 10, and a monitor 3 and an input unit 4 connected to the computer 2.

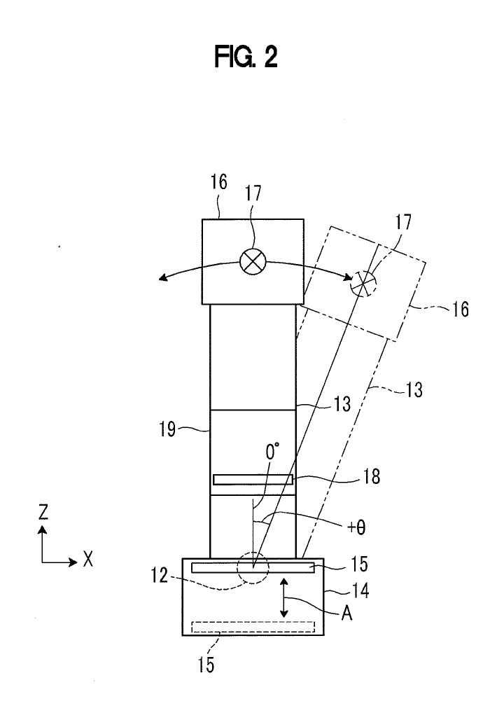

As shown in FIG. 1, the breast image radiographing apparatus 10 includes a base 11, a rotating shaft 12 that can move relative to the base 11 in the vertical direction (Z direction) and that can rotate, and an arm section 13 connected to the base 11 via the rotating shaft 12. FIG. 2 shows the arm section 13 as viewed from the right side in FIG. 1.

The arm section 13 has the shape of letter C, has a radiography platform 14 attached to one end and a radiation irradiating unit 16 attached to the other end so as to face the radiography platform 14. The rotation and the movement in the vertical direction of the arm section 13 are controlled by an arm controller 31 built in the base 11.

The inside of the radiography platform 14 is provided with a radiological image detector 15 such as a flat panel detector, a distance changing mechanism 14a that changes the distance between the radiological image detector 15 and the breast M located on the radiography platform 14 by moving the radiological image detector 15 in the direction of arrow A shown in FIG. 1, and a detector controller 33 that controls the movement of the radiological image detector 15 by the distance changing mechanism 14a and the reading of a charge signal from the radiological image detector 15.

The inside of the radiography platform 14 is also provided with a circuit board or the like in which a charge amplifier converting the charge signal read from the radiological image detector 15 into a voltage signal, a correlation double sampling circuit sampling the voltage signal output from the charge amplifier, an AD converter converting the voltage signal into a digital signal, and the like are disposed.

The radiography platform 14 is configured to be rotatable about the arm section 13. When the arm section 13 rotates relative to the base 11, the direction of the radiography platform 14 can be fixed relative to the base 11.

The radiological image detector 15 can repeatedly write and read the radiological images and may employ a so-called direct-type radiological image detector being directly subjected to the irradiation with radiation and generating charges or may employ a so-called indirect-type radiological image detector converting radiation into a visible ray and converting the visible ray into a charge signal. A so-called a TFT reading method of reading a radiological image signal by turning on and off a TFT (Thin Film Transistor) or a so-called optical reading method of reading a radiological image signal by applying a reading light beam can be preferably used to read the radiological image signal, but other methods may be employed without being limited to these methods.

The distance changing mechanism 14a changes the distance between the radiological image detector 15 and the breast M located on the radiography platform 14 as described above, and this change in distance is performed by switching the radiographing mode between a normal radiographing mode and an enlarged radiographing mode.

The normal radiographing mode in this embodiment is a mode in which the radiographing operation is performed in a state where the radiological image detector 15 is closest to the breast M. Here, it is assumed that a radiological image with a magnification approximately equal to the breast M is projected onto the radiological image detector 15. The enlarged radiographing mode is a mode in which the radiographing operation is performed in a state where the radiological image detector 15 is more separated from the breast M than in that normal radiographing mode. Here, a radiological image of the breast M enlarged at an enlarged ratio larger than the equal magnification by increasing the distance between the radiological image detector 15 and the breast M is projected onto the radiological image detector 15.

The enlargement ratio in the enlarged radiographing mode can be set to any value input from a radiographer through the use of the input unit 4. The distance changing mechanism 14a moves the radiological image detector 15 by a distance corresponding to the set enlargement ratio. Specifically, for example, when the enlargement ratio is n (n>1), the distance changing mechanism 14a moves the radiological image detector 15 so that the distance from the focal point of the radiation source 17 to the detection plane of the radiological image detector 15 is n times the distance in the normal radiographing mode.

A radiation source 17 and a radiation source controller 32 are received in the radiation irradiating unit 16. The radiation source controller 32 can control the time to radiate from the radiation source 17 and radiation generating conditions (such as the tube current, the time, and the product of tube current and time) in the radiation source 17.

The central portion of the arm section 13 is provided with a compression plate 18 being disposed above the radiography platform 14 and pressing a breast, a support 20 supporting the compression plate 18, and a moving mechanism 19 moving the support 20 in the vertical direction (Z direction). The position and the pressing pressure of the compression plate 18 are controlled by a compression plate controller 34.

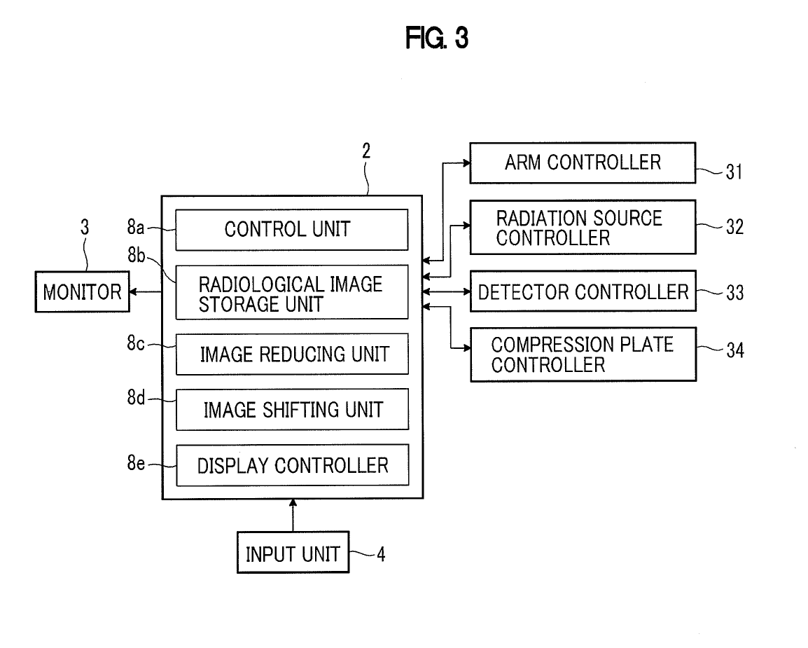

The computer 2 includes a central processing unit (CPU) and a storage device such as a semiconductor memory, a hard disk, or an SSD. A control unit 8a, a radiological image storage unit 8b, an image reducing unit 8c, an image shifting unit 8d, and a display controlling unit 8e shown in FIG. 3 are constructed by these hardware components.

The control unit 8a outputs a predetermined control signal to various controllers 31 to 35 and controls the entire system. The specific control method will be described later in detail.

The radiological image storage unit 8b stores two radiological image signals detected by radiographing an image from two different radiographing directions by the use of the radiological image detector 15 in advance.

The image reducing unit 8c performs a reduction process on two radiological image signals read from the radiological image storage unit 8b based on the enlargement ratio in the enlarged radiographing mode. Specifically, when the enlargement ratio in the enlarged radiographing mode is n (n>1), the image reducing unit 8c performs a 1/n reduction process on two radiological image signals. A known method such as a thinning-out process can be employed as the method of performing the reduction process.

The image shifting unit 8d performs a shifting process of relatively shifting two radiological images having been subjected to the reduction process by the image reducing unit 8c in the parallax direction between two radiological images based on the angle formed by the radiographing directions of two radiological images constituting a stereo image and the enlargement ratio in the enlarged radiographing mode. The amount of shift in the shifting process will be described later in detail.

In the normal radiographing mode, the display controlling unit 8e performs a predetermined process on two radiological image signals read from the radiological image storage unit 8b and then displays the stereo image, which is obtained by radiographing the breast M in the normal radiographing mode, on the monitor 3. In the enlarged radiographing mode, the display controlling unit performs a predetermined process on two radiological image signals having been subjected to the shifting process by the image shifting unit 8d and then displays the stereoscopic image, which is obtained by radiographing the breast M in the enlarged radiographing mode, on the monitor 3.

The input unit 4 includes pointing devices such as a keyboard or a mouse and receives the input of radiographing conditions including the enlargement ratio, the input of a radiographing start instruction, or the like from a radiographer.

The monitor 3 is configured to display a stereo image using two radiological image signals output from the computer 2 at the time of radiographing a stereo image. As the configuration for displaying a stereo image, a configuration for displaying a stereo image by displaying the radiological images based on two radiological image signals, for example, using two screens, allowing one radiological image to be incident on an observer's right eye using a half mirror or a polarizing glass and allowing the other radiological image to be incident on the observer's left eye can be employed. Alternatively, a stereo image may be generated by dislocating, superimposing and displaying two radiological images by a predetermined magnitude of parallax and observing the resultant image with a polarizing glass may be employed, or a stereo image may be generated by displaying two radiological images by the use of a 3D liquid crystal display enabling a stereoscopic view like a parallax barrier method or a lenticular method.

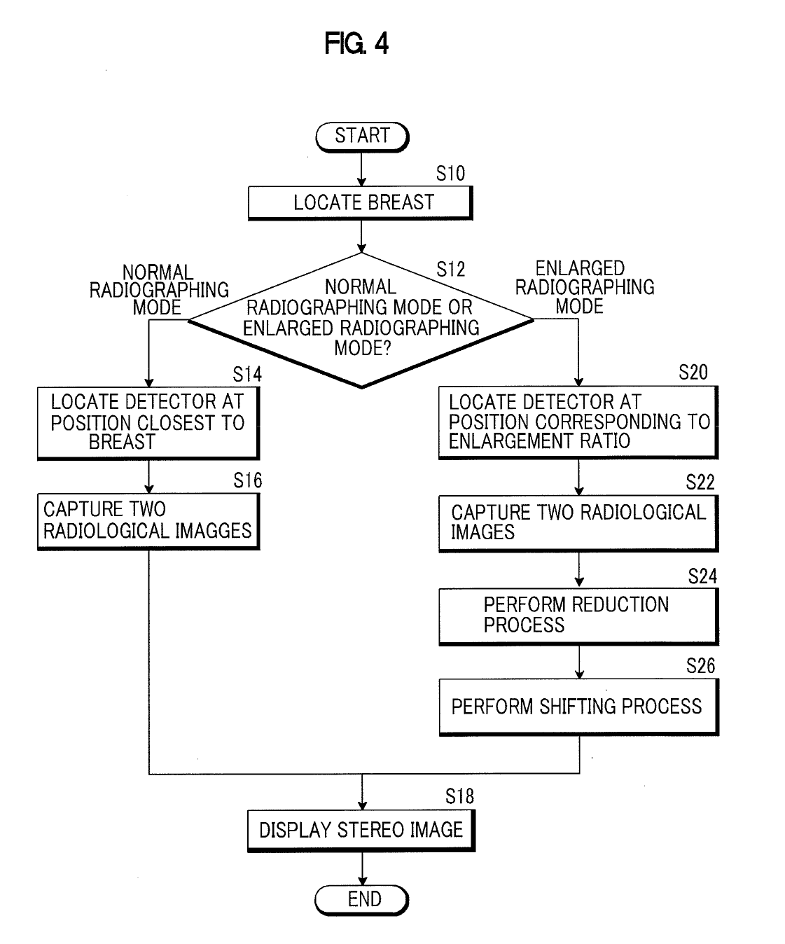

The operation of the breast image radiographing and displaying system according to this embodiment will be described with reference to the flowchart shown in FIG. 4.

First, a patient's breast M is located on the radiography platform 14 and the breast M is pressed with a predetermined pressure by the compression plate 18 (S10).

A radiographer inputs various radiographing conditions along with a selection instruction of the normal radiographing mode or the enlarged radiographing mode and then inputs a radiographing start instruction through the use of the input unit 4 (S12).

When the selection instruction of the normal radiographing mode and the radiographing start instruction are input to the input unit 4, the radiological image detector 15 is located at the position closest to the breast M by the distance changing mechanism 14a (S14) and the first radiological image out of two radiological images constituting the stereo image of the breast M is captured in this state (S16).

Specifically, first, the control unit 8a reads a preset convergence angle θ for radiographing a stereoscopic image and outputs the information of the read convergence angle θ to the arm controller 31. In this embodiment, it is assumed that θ=4° is stored in advance as the information of the convergence angle θ, but the present invention is not limited to this configuration and any convergence angle can be set by the use of the input unit 4.

When the arm controller 31 receives the information of the convergence angle θ output from the control unit 8a, the arm controller 31 outputs a control signal so that the arm section 13 is perpendicular to the radiography platform 14, as shown in FIG. 2.

In the state where the arm section 13 is located perpendicular to the radiography platform 14 based on the control signal output from the arm controller 31, the control unit 8a outputs a control signal to the radiation source controller 32 and the detector controller 33 so as to radiate and to read a radiological image signal. In response to this control signal, radiation is radiated from the radiation source 17, the radiological image obtained by radiographing the breast in the direction of 0° is detected by the radiological image detector 15, the radiological image signal is read by the detector controller 33, the read radiological image signal is subjected to a predetermined signal process, and the resultant signal is stored in the radiological image storage unit 8b of the computer 2.

Then, as shown in FIG. 2, the arm controller 31 outputs a control signal so that the arm section 13 rotates by +θ° about the direction perpendicular to the radiography platform 14. That is, in this embodiment, the control signal is output so that the arm section 13 rotates by 4° about the direction perpendicular to the radiography platform 14.

In the state where the arm section 13 rotates by 4° in response to the control signal output from the arm controller 31, the control unit 8a outputs a control signal to the radiation source controller 32 and the detector controller 33 so as to radiate and to read a radiological image signal.

Specifically, the control unit 8a outputs a control signal to the radiation source controller 32 and the detector controller 33 so as to radiate and to read a radiological image. In response to this control signal, the radiation is emitted from the radiation source 17, the radiological image obtained by radiographing the breast in the direction of 4° is detected by the radiological image detector 15, the radiological image signal is read by the detector controller 33, the read radiological image signal is subjected to a predetermined signal process, and the resultant signal is stored in the radiological image storage unit 8b of the computer 2.

Two radiological image signals stored in the radiological image storage unit 8b are readout, a predetermined process is performed on the read radiological image signals by the display controlling unit 8e, the resultant signal are output to the monitor 3, and a stereo image of the breast in the normal radiographing mode is displayed on the monitor 3 (S18).

On the other hand, when the radiographing start instruction along with the selection instruction of the enlarged radiographing mode is input to the input unit 4, the radiological image detector 15 is located at the position corresponding to the enlargement ratio by the distance changing mechanism 14a (S20) and two radiological images constituting the stereos image of the breast M are captured in this state (S22). The enlargement ratio in the enlarged radiographing mode may be set in advance or may be set to any value by the radiographer through the use of the input unit 4.

The radiographing of two radiological images is performed similarly to the above-mentioned normal radiographing mode. The radiological image obtained by radiographing the breast in the direction of 0° and the radiological image obtained by radiographing the breast in the direction of 4° are detected by the radiological image detector 15 and these two radiological image signals are stored in the radiological image storage unit 8b.

Two radiological image signals stored in the radiological image storage unit 8b are read out and are input to the image reducing unit 8c, and the reduction process corresponding to the enlargement ratio is performed on two radiological image signals by the image reducing unit 8c (S24).

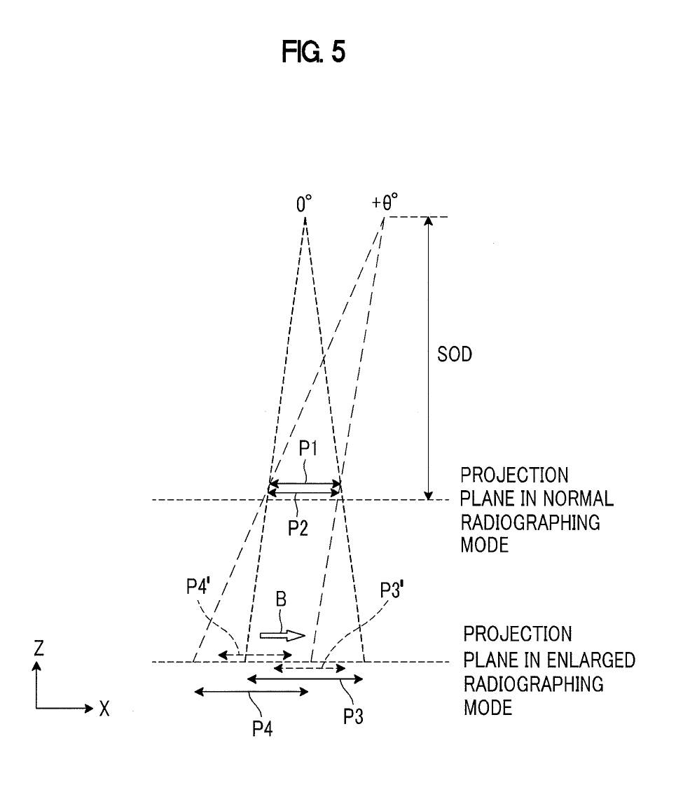

Here, FIG. 5 is a diagram schematically illustrating two radiological images P1 and P2 projected onto the detection plane (projection plane) of the radiological image detector 15 in the normal radiographing mode, two radiological images P3 and P4 projected onto the detection plane (projection plane) of the radiological image detector 15 in the enlarged radiographing mode, and two radiological images P3′ and P4′ obtained by performing the reduction process on two radiological images P3 and P4 captured in the enlarged radiographing mode.

As shown in FIG. 5, in the enlarged radiographing mode, since the detection plane (projection plane) of the radiological image detector 15 is more separated from the breast than in the normal radiographing mode, the radiological image enlarged by the separated distance is projected onto the detection plane of the radiological image detector 15. In this embodiment, the enlarged radiological image is subjected to the reduction process so as to have the same size as the radiological image captured in the normal radiographing mode. That is, in this embodiment, the equal-magnification radiographing operation is performed in the normal radiographing mode as described above. Accordingly, when the enlargement ratio in the enlarged radiographing mode is n, the 1/n reduction process is performed on two radiological images.

Two radiological image signals having been subjected to the reduction process by the image reducing unit 8c are input to the image shifting unit 8d and are subjected to the shifting process by the image shifting unit 8d (S26). Specifically, in this embodiment, the shifting process of shifting the radiological images in the direction of arrow B is performed on the radiological images P4′ having been subjected to the reduction process, as shown in FIG. 5, so that the magnitude of parallax between the radiological image P1 and the radiological image P2 captured in the normal radiographing mode is equal to the magnitude of parallax between the radiological image P3′ and the radiological image P4′ having been subjected to the reduction process. Accordingly, the image with the edge enhancement resulting from the X-ray interference effect caused by appropriately setting the distance between the radiation source and the radiological image detector as a secondary effect can be viewed stereoscopically with an appropriate magnitude of parallax.

Here, the amount of shift S of the shifting process performed on the radiological image P4′ is calculated by the use of following expression.

S=(n−1)×tanθ

In the expression, n represents the enlargement ratio, SOD represents the distance from a focal point of radiation in the enlarged radiographing mode to the detection plane (projection plane) of the radiological image detector 15 in the normal radiographing mode as shown in FIGS. 5, and θ represents the convergence angle and is 4° in this embodiment.

In this embodiment, the shifting process is performed on only the radiological image P4′. However, the shifting process may be performed on only the radiological image P3′ or on both the radiological image P3′ and the radiological image P4′ so that the relative amount of shift is S.

The radiological image signal having been subjected to the shifting process by the image shifting unit 8d is read, the read radiological image signal is subjected to a predetermined process by the display controlling unit 8e and is then output to the monitor 3, and the stereo image obtained by radiographing the breast in the enlarged radiographing mode is displayed on the monitor 3 (S18).

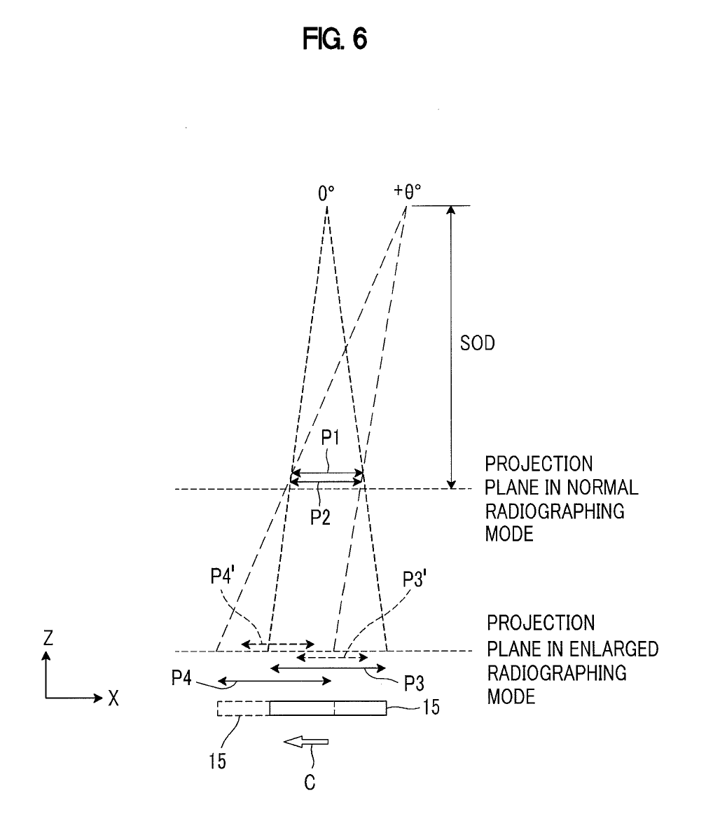

In the embodiment, the magnitude of parallax between two radiological images in the enlarged radiographing mode is made to be equal to the magnitude of parallax between two radiological images in the normal radiographing mode by performing the shifting process by the use of the image shifting unit 8d, but the present invention is not limited to this configuration. For example, instead of performing the shifting process, the radiological image detector 15 may be moved in the parallax direction (in the X direction) by the distance changing mechanism 14a, as shown in FIG. 6.

Specifically, for example, as shown in FIG. 6, the radiological image detector 15 may be moved in the direction of arrow C by the amount of shift S from the position at which the radiological image P3′ is captured in the direction of 0° when radiographing the radiological image P4′ in the direction of 4°. The method of calculating the amount of shift S is the same as performing the shifting process.

In the above description, the radiological image detector 15 is moved only when the radiological image P4′ is captured, but the present invention is not limited to this configuration. The radiological image detector 15 may be moved only when the radiological image P3′ is captured or the radiological image detector 15 may be moved both when the radiological image P3′ is captured and when the radiological image P4′ is captured, so that the relative amount of shift S is obtained.

In the embodiment, the magnitude of parallax between two radiological images in the enlarged radiographing mode is made to be equal to the magnitude of parallax between two radiological images in the normal radiographing mode by performing the shifting process by the use of the image shifting unit 8d. However, instead of performing the shifting process on two radiological image signals, at the time of displaying a radiological image based on two radiological image signals on the monitor 3, the display controlling unit 8e may generate a display control signal for relatively shifting and displaying two radiological images in the parallax direction based on the angle formed by two radiographing directions and the enlargement ratio and may output the generated display control signal to the monitor 3 so as to display two radiological images, so that the magnitude of parallax between two radiological images in the enlarged radiographing mode is equal to the magnitude of parallax between tow radiological images in the normal radiographing mode. It is most preferable that the magnitudes of parallax between the images are equal to each other, but the magnitudes of parallax between the images may have a approximately equal magnification. Here, the approximately equal magnification means that the difference in the magnitude of parallax between the images is preferably ±20% or less and that the difference in the magnitude of parallax between the images is more preferably ±10% or less.

In the above description, the radiological image radiographing apparatus according to an embodiment of the present invention is applied to the breast image radiographing and displaying system. However, the subject in the present invention is not limited to the breast and the present invention can be applied to a radiological image radiographing and displaying system radiographing, for example, a chest region or a head region.

What is claimed is:

1. A radiological image radiographing and displaying method of irradiating a subject with radiation radiated from two focal positions from two different radiation directions, detecting a radiological image in each radiographing direction resulting from the irradiation with the radiation by the use of a radiological image detector, and displaying a stereoscopic image using the two detected radiological images, and the distance between the subject and the radiological image detector is changed depending on an enlargement ratio of the radiological images to detect the radiological images, wherein the method consists of the steps of
relatively moving two radiological images in a parallax direction which is obtained by projecting an extending direction of a straight line connecting the two focal positions onto the corresponding radiological image, and then displaying the moved two radiological images, based on the angle formed by the two radiographing directions and the enlargement ratio.

2. A radiological image radiographing and displaying method of irradiating a subject with radiation radiated from two focal positions from two different radiographing directions, detecting a radiological image in each radiographing direction resulting from the irradiation with the radiation by the use of a radiological image detector, and displaying a stereoscopic image using the two detected radiological images, wherein the method consists of the steps of
changing the distance between the subject and the radiological image detector depending on an enlargement ratio of the radiological images to detect the radiological images, and
relatively moving the radiological image detector moved in a parallax direction, which is obtained by projecting an extending direction of a straight line connecting the two focal positions onto the corresponding radiological image, based on the angle formed by the two radiographing directions and the enlargement ratio, to capture the radiological images.

3. A radiological image radiographing and displaying apparatus comprising:
a radiation irradiating unit that irradiates a subject with radiation radiated from two focal positions from two different radiographing directions;
a radiological image detector that detects a radiological image in each radiographing direction resulting from the irradiation with the radiation from the radiation irradiating unit;
a distance changing mechanism that changes the distance between the subject and the radiological image detector depending on an enlargement ratio of the radiological images; and
a display unit that displays a stereoscopic image using two radiological images detected by the radiological image detector,
the display unit relatively moves the two radiological images in a parallax direction which is obtained by projecting an extending direction of a straight line connecting the two focal positions onto the corresponding radiological image based on the angle formed by the two radiographing directions and the enlargement ratio and the display unit displays the moved two radiological images.

4. A radiological image radiographing and displaying apparatus comprising:
a radiation irradiating unit that irradiates a subject with radiation radiated from two focal positions from two different radiographing directions;
a radiological image detector that detects a radiological image in each radiographing direction resulting from the irradiation with the radiation from the radiation irradiating unit;
a distance changing mechanism that changes the distance between the subject and the radiological image detector depending on an enlargement ratio of the radiological images;
a display unit that displays a stereoscopic image using two radiological images detected by the radiological image detector; and
a moving mechanism that relatively moves the radiological image detector in a parallax direction which is obtained by projecting an extending direction of a straight line connecting the two focal positions onto the corresponding radiological image based on the angle formed by the two radiographing directions and the enlargement ratio.

5. The radiological image radiographing and displaying apparatus according to claim 3, further comprising an image shifting unit that performs a shifting process of relatively shifting the two radiological images in the parallax direction based on the angle formed by the two radiographing directions and the enlargement ratio.

6. The radiological image radiographing and displaying apparatus according to claim 3, further comprising a display control unit that controls the relative shifting and displaying of the two radiological images in the parallax direction based on the angle formed by the two radiographing directions and the enlargement ratio when displaying the two radiological images on the display unit.

7. The radiological image radiographing and displaying apparatus according to claim 3, further comprising an image reducing unit that increases a reduction ratio of the two radiological images with the increasing of the enlargement ratio.

8. The radiological image radiographing and displaying apparatus according to claim 4, further comprising an image reducing unit that performs a reduction process on the two radiological images based on the enlargement ratio.

9. The radiological image radiographing and displaying apparatus according to claim 5, further comprising an image reducing unit that performs a reduction process on the two radiological images based on the enlargement ratio.

10. The radiological image radiographing and displaying apparatus according to claim 6, further comprising an image reducing unit that performs a reduction process on the two radiological images based on the enlargement ratio.

11. The radiological image radiographing and displaying apparatus according to claim 5, wherein the image shifting unit performs the shifting process so that the magnitude of parallax between the two radiological images when the enlargement ratio is larger than 1 approaches the magnitude of parallax between the two radiological images when the enlargement ratio is approximately an equal magnification.

12. The radiological image radiographing and displaying apparatus according to claim 6, wherein the display controlling unit controls the relative shifting and displaying of the two radiological images so that the magnitude of parallax between the two radiological images when the enlargement ratio is larger than 1 approaches the magnitude of parallax between the two radiological images when the enlargement ratio is approximately an equal magnification.

13. The radiological image radiographing and displaying apparatus according to claim 4, wherein the moving mechanism moves the radiological image detector so that the magnitude of parallax between the two radiological images when the enlargement ratio is larger than 1 approaches the magnitude of parallax between the two radiological images when the enlargement ratio is approximately an equal magnification.

14. The radiological image radiographing and displaying apparatus according to claim 3, further comprising a radiation mode instruction receiving unit that receives an instruction of a normal radiation mode in which the enlargement ratio is approximately an equal magnification and an enlarged radiographing mode in which the enlargement ratio is larger than 1.

15. The radiological image radiographing and displaying apparatus according to claim 4, further comprising a radiographing mode instruction receiving unit that receives an instruction of a normal radiographing mode in which the enlargement ratio is approximately an equal magnification and an enlarged radiographing mode in which the enlargement ratio is larger than 1.

16. The radiological image radiographing and displaying apparatus according to claim 3, wherein the distance changing mechanism moves the radiological image detector relative to the subject.

17. The radiological image radiographing and displaying apparatus according to claim 4, wherein the distance changing mechanism moves the radiological image detector relative to the subject.

18. The radiological image radiographing and displaying apparatus according to claim 5, wherein the distance changing mechanism moves the radiological image detector relative to the subject.

19. The radiological image radiographing and displaying apparatus according to claim 6, wherein the distance changing mechanism moves the radiological image detector relative to the subject.

20. The radiological image radiographing and displaying apparatus according to claim 7, wherein the distance changing mechanism moves the radiological image detector relative to the subject.
